(19) United States
(12) Patent Application Publication
(10) Pub. No.: US 2005/0214243 A1
(43) Pub. Date: Sep. 29, 2005

(54) Feminine homeopathic formulations

(76) Inventor: Jason Fleming, Nevada City, CA (US)

Correspondence Address: FISH & RICHARDSON P.C., PO BOX 1022, MINNEAPOLIS, MN, 55440-1022, US

(21) Appl. No.: 11/090,113

(22) Filed: Mar. 24, 2005

Related U.S. Application Data
(60) Provisional application No. 60/556,487, filed on Mar. 26, 2004.

(51) Int. Cl. A61K 7/06; A61K 35/78; A61K 33/06

(52) U.S. Cl. 424/74; 424/725; 424/195.16; 424/770; 424/726; 424/696

(57) ABSTRACT

The formulations of the present invention include personal lubricants and topical applications, preferably made from substantially wholly organic ingredients, which include homeopathic ingredients which are known to be useful to support the female reproductive system. The lubricants are preferably water-based. In a preferred embodiment the lubricants are rehydrating.

### CROSS-REFERENCE TO RELATED APPLICATIONS

This application claims the benefit under 35 U.S.C. § 119(e) of U.S. Provisional Application Ser. No. 60/556,487, filed on Mar. 26, 2004. The disclosure of the prior application is considered part of and is incorporated by reference in its entirety in the disclosure of this application.

### BACKGROUND

This invention relates to formulations for topical application and lubricants, and more particularly to personal lubricants useful for sexual intercourse and methods for their use. The lubricants can be water-based and contain natural, organic ingredients, including homeopathic ingredients.

The use of personal lubricants as an aid to sexual intercourse is known in the art. However, previous personal lubricant compositions were generally based on petroleum distillates and other non-organic components which can cause adverse reactions such as allergies or irritate sensitive tissues.

Homeopathic remedies are known to restore balance in the body and stimulate the natural healing power of the body. See, e.g., Homeopathic Remedy Guide by Robin Murphy ND (2000) and Homeopathic Medical Repertory by Robin Murphy ND Second Edition (2003). Generally, homeopathic remedies are administered orally to neutralize disorders, including disorders relating to male and female sexuality.

Intimate contact between individuals, such as during sexual intercourse, often suffers from an undesirably high amount of friction between the individuals. It is known in the prior art to provide various personal lubricants to reduce the coefficient of friction exhibited between the individuals to enhance the comfort and pleasure associated with the experience. One common category of such lubricants are lubricants formed from petroleum distillates, such as glycerine mixed with a water base. Various products are known to be provided in this category, which typically include various other ingredients in their particular formulations to distinguish them from each other.

Glycerine is known to be an effective lubricant and to be water soluble and generally acceptable for use directly on human skin. However, this and other petroleum distillate derived personal lubricants, such as other glycerine based lubricants, suffer from numerous drawbacks.

Specifically, such products are not organically derived, but rather manufactured through chemical engineering techniques, typically from crude oil or other petroleum products. Thus, such products suffer from all of the known, and potential unknown consequences resulting from the use of such products. Many individuals and experts have come to recognize that when products are provided which are derived from organic sources, rather than manufactured from petroleum products, that the effectiveness of such products is enhanced.

Further, oil-containing personal lubricants can cause rapid deterioration of latex condoms. As little as one minute's exposure to mineral oil caused approximately 90% decrease in the strength of condoms. (Voeller et al., Contraception 39:95-102 (1989)).

Additionally, some individuals can experience allergies or other undesirable reactions when utilizing products distilled from petroleum while not having corresponding detrimental reactions to organically derived products. Finally, many individuals recognize the benefits of the lesser impact on the environment and the benefits of sustainable development associated with organic products. While these benefits alone suggest the desirability for the providing of personal lubricants made from materials other than petroleum distillates, such petroleum distillate lubricants also can provide a less than wholly satisfactory function as a personal lubricant.

Particularly, these petroleum distillate personal lubricants experience particular negative attributes in many circumstances. Such petroleum distillate personal lubricants can experience a dehydration phenomena where, as the petroleum distillate personal lubricant formula undergoes evaporation or selective absorption such that the lubricant begins to dry out, the lubricant undergoes a radical increase in coefficient of friction, changing from slick to sticky. This phenomenon can be particularly recognized by merely placing petroleum distillate based personal lubricants on ones hands and rubbing ones hands together for about two minutes. The petroleum distillate formulation will dry out and become sticky, actually exacerbating the friction problem rather than remediating it. When water is added to the water based petroleum distillate personal lubricants they do return from sticky to slick. While this return to slickness does last for a few minutes, further dehydration causes the personal lubricant to again attain a sticky character.

Further rehydration may fail to renew the petroleum distillate based personal lubricants. Rather, they attain a coefficient of friction similar to that of water when sufficient rehydration has occurred, such that the petroleum distillate based personal lubricants lose their effectiveness after about five minutes. Often such a minimal duration of function is unacceptable as the period of intimate contact has not yet ceased, resulting in discomfort or disruption for reapplication of additional lubricant.

Accordingly, a need exists for a personal lubricant which is derived from organic sources and contains homeopathic ingredients which can provide enhanced health benefits. In particular, a homeopathic-containing personal lubricant which is derived from organic sources rather than from petroleum distillates, maintains its slipperiness for as long a duration as possible, and is readily rehydratable with liquids secreted by the individuals during intercourse, such that slipperiness is maintained for a long period of time without requiring rehydration would be desirable.

### SUMMARY

The formulations of the present invention are for topical application and/or for lubrication, preferably made from substantially wholly organic ingredients, which include homeopathic ingredients which are known to be useful to support the health of the female reproductive system. The formulations can be water-based. In one embodiment the formulations are lubricants that are rehydrating, as described in U.S. patent application Ser. No. 10/795,172, filed Mar. 4, 2004, the disclosure of which is incorporated herein by reference in its entirety. Such lubricants contain guar gum and exhibit native rehydration when in use. The compositions and formulations of the present invention have a low coefficient of friction when applied to mating surfaces, such that they can function effectively as lubricants and especially as personal lubricants such as for sexual intercourse.

Accordingly, in one aspect the invention provides a personal lubricant composition comprising at least one component selected from the group consisting of pulsatilla, berberis vulgaris, hepar-sulphuris-calcareum, lilium tigrinum, hydrastis canadensis, cantharis, candida albicans, sepia, thuja occidentalis and combinations thereof. The lubricant may be water-based. The lubricant may be substantially all natural and/or all organic. In particular, lubricants containing at least one of sepia or thuja occidentalis and combinations thereof are useful to support the female reproductive system.

In another aspect, the invention provides a rehydrating water-based personal lubricant composition comprising guar gum and at least one component selected from the group consisting of pulsatilla, berberis vulgaris, hepar-sulphuris-calcareum, lilium tigrinum, nydrastis canadensis, cantharis, candida albicans, sepia, thuja occidentalis and combinations thereof. The lubricant may preferably be substantially all natural and/or all organic. In particular, lubricants containing at least one of sepia or thuja occidentalis and combinations thereof are useful to support the female reproductive system.

In yet another aspect, the invention provides a rehydrating water-based personal lubricant composition comprising guar gum, at least one component selected from material derived from an aloe vera plant, grapefruit seed extract and vitamin E, and at least one component selected from the group consisting of pulsatilla, berberis vulgaris, hepar-sulphuris-calcareum, lilium tigrinum, hydrastis canadensis, cantharis, candida albicans, sepia, thuja occidentalis and combinations thereof. The lubricant may preferably be substantially all natural and/or all organic. In particular, lubricants containing at least one of sepia or thuja occidentalis and combinations thereof are useful to support the female reproductive system.

In yet a further aspect, the invention provides a rehydrating water-based personal lubricant composition comprising material derived from an aloe vera plant, grapefruit seed extract, vitamin E, guar gum and at least one component selected from the group consisting of pulsatilla 6c, berberis vulgaris 6c, hepar-sulphuris-calcareum 6c, lilium tigrinum 6c, hydrastis canadensis 6c, cantharis 6c, candida albicans 6c, sepia 6c, thuja occidentalis 6c and combinations thereof. The lubricant may preferably be substantially all natural and/or all organic. In particular, lubricants containing at least one of sepia 6c or thuja occidentalis 6c and combinations thereof are useful to support the female reproductive system.

In one aspect, the invention provides a composition for topical application comprising at least one component selected from the group consisting of pulsatilla, berberis vulgaris, hepar-sulphuris-calcareum, lilium tigrinum, hydrastis canadensis, cantharis, candida albicans, sepia and thuja occidentalis. The composition can include substantially all natural and/or all organic ingredients and components.

In any of the compositions described above, one or more of a material derived from an aloe vera plant, grapefruit seed extract or vitamin E can be included in the composition.

In still another aspect, the invention provides methods of using the personal lubricant compositions described above by applying the lubricant to a female individual prior to sexual activity. Alternatively, the lubricant can be applied to a female individual during daily activities outside of sexual activity.

In yet another aspect, the invention provides methods of using the composition for topical application. The composition can be applied to support the female reproductive system.

In one aspect, one or more homeopathic ingredients, such as pulsatilla, berberis vulgaris, hepar-sulphuris-calcareum, lilium tigrinum, hydrastis canadensis, cantharis, candida albicans, sepia, thuja occidentalis, can be incorporated into water-based or non-water based formulations, such as glycerine-, silicon-, or petroleum-based formulations. The formulation can be one that is appropriate for applying to the body, such as a cream, lotion, gel, emollient, balm, liquid or oil.

The invention can include none, one or more of the following advantages. The formulation can be water-based and may be used in combination with latex- or silicone-based materials. The formation may be rehydrating and may be rehydrated by water-based fluids for an indefinite length of time. The slippery nature of the formulation may provide a desired reduction in friction between a body part and another object, such as another body part or a device. Reducing friction caused on a body part can reduce an amount of inflammation, pain, burning or itching sensation that may be caused, such as by sexual activity involving the body part. The ingredients of the formation may be such that the formulation is safe for ingestion.

Sexual activity can lead to burning, itching, discharge, dryness, swelling, tissue tenderness, bladder infections and candida infections. Prior art lubricants do not necessarily address these problems. The use of homeopathic ingredients may prevent irritation and infection from occurring. If homeopathic ingredients that are useful for supporting the health of the female reproductive system are included in the formulation, the homeopathic ingredients may reduce inflammation, pain, burning or itching sensations experienced by the user. Further, topical application of the homeopathic ingredients may prevent or alleviate the symptoms of some types of vaginal discharge, bladder infection or candida overgrowth. Formations, such as lotions, ointments, oils, creams, balms or other topical applications including one or more of the homeopathic ingredients can be applied to support the female reproductive system. The formulation can be applied to improve the general wellbeing of a subject and decrease discomfort due to vaginal dryness.

The details of one or more embodiments of the invention are set forth in the description below. Other features, objects, and advantages of the invention will be apparent from the description, and from the claims.

### DETAILED DESCRIPTION

This invention is directed to formulations which support the female reproductive system. The formulations are preferably water-based and made from substantially wholly organic ingredients. They include homeopathic ingredients known to be useful to support the female reproductive system and the health of the female reproductive system. Homeopathy uses a wide variety of animal, plant, and inorganic substances. Homeopathic components for use in treating a disease or supporting the health of a subject can be selected according to components that in larger doses would case similar symptoms to those of the disease to be treated or avoided in a healthy person. The ingredients may actively stimulate healthy body systems, including the immune system. The formulations can include lubricants. In a preferred embodiment the lubricants are rehydrating, as described in U.S. patent application Ser. No. 10/795,172, filed Mar. 4, 2004, the disclosure of which is incorporated herein by reference in its entirety, which describes lubricants containing guar gum and exhibiting native rehydration when in use. The lubricant compositions and formulations of the present invention can have a low coefficient of friction when applied to mating surfaces, such that they function effectively as lubricants and especially as personal lubricants such as for sexual intercourse. In addition, they contain homeopathic components which support the health of the female reproductive system.

The personal lubricants of the present invention comprise homeopathic ingredients useful to support the female reproductive system in a lubricant base. A water-based lubricant is preferred, with rehydrating water-based lubricants being especially preferred. In one embodiment, the rehydrating water-based personal lubricant composition comprises material derived from an aloe vera plant, grapefruit seed extract, vitamin E and guar gum. See U.S. patent application Ser. No. 10/795,172, filed Mar. 4, 2004.

The water-based personal lubricant compositions of the present invention also comprise at least one homeopathic component selected from the group consisting of pulsatilla, berberis vulgaris, hepar-sulphuris-calcareum, lilium tigrinum, hydrastis canadensis, cantharis, candida albicans, sepia, thuja occidentalis and combinations thereof. These homeopathic ingredients may be purchased, for example, from Dolisos Homeopathic Remedies. (http://www.zhealthinfo.com/dolisos.htm) Various strengths may be used, but the preferred strength of these homeopathic components is: pulsatilla 6c, berberis vulgaris 6c, hepar-sulphuris-calcareum 6c, lilium tigrinum 6c, hydrastis canadensis 6c, cantharis 6c, candida albicans 6c, sepia 6c, thuja occidentalis 6c. In particular, it has been found that lubricants containing at least one of sepia and thuja occidentalis and combinations thereof are especially useful to support the female reproductive system.

Each homeopathic component used may be present in the lubricant composition in an amount ranging from about 0.0001% to about 10%, by volume. Certain embodiments may contain from about 0.001% to about 1% (by volume) of each homeopathic component that is present in the composition. In further embodiments, a homeopathic component may comprise from about 0.01% to about 0.1% (v/v) of the composition. In one embodiment, about 0.02% to about 0.03% by volume of each homeopathic component is used.

All possible combinations of the homeopathic components may be used in the formulations of the present invention. That is to say, a formulation of the present invention may contain one, two, three, four, five, six, seven, eight or nine of the homeopathic components pulsatilla, berberis vulgaris, hepar-sulphuris-calcareum, lilium tigrinum, hydrastis canadensis, cantharis, candida albicans, sepia and thuja occidentalis in any combination.

The constituents which make up a water-based rehydrating personal lubricant base useful in this invention according to its preferred embodiments are guar gum and water or other water containing solvent sufficient to cause the guar gum to form a gel. The water containing solvent is preferably deionized water. The ratio of guar gum to water containing solvent is preferably between about 1% and about 2%. However, if the amount of the formulation provided by guar gum is between about 0.01% and about 15%, many of the benefits of this invention can be achieved, with formulations having an amount of guar gum between about 1.0% and about 10% considered to be within a most desirable range.

Many of the benefits of the personal lubricant of this invention can be provided by merely combining the guar gum with a water containing solvent as identified above. The water containing solvent can be water alone, and preferably deionized water. This water containing solvent can also be aloe vera juice or a combination of aloe vera juice and water. This water containing solvent can also be other water containing solvents capable of converting the guar gum from a paste consistency into a gel.

The composition derives further benefits from including other constituents as follows. The composition preferably includes both water in the form of deionized water and material from the aloe vera plant, most preferably in the form of aloe vera juice. Aloe vera juice is particularly provided by filleting the leaves of the aloe vera plant and squeezing/pressing them to extract the “juice.” The aloe vera juice is preferably provided in a range between about 0% and about 90%. Any amount between about 70% and about 90% is considered most desirable, with about 80% considered to be optimal.

The preferred formulation additionally can include grapefruit seed extract. This grapefruit seed extract is preferably provided as between about 0.001% and about 5% of the entire formulation. Most desirably the grapefruit seed extract is provided in an amount between about 0.01% and about 0.5% with about 0.10% to about 0.15% considered most optimal.

The preferred formulation can include vitamin E as a natural preservative. The vitamin E additionally provides associated health benefits known to be associated with vitamin E. The vitamin E is preferably provided as between about 0% and about 5% of the entire formulation. Amounts of vitamin E between about 1.0% and about 3.0% are considered most desirable, with about 2.3% to about 2.8% considered optimal.

Additional constituents can additionally be provided within the formulation if desired, such as flavorings, fragrances, aromas, medicinals, colorants, etc.

The process of mixing the constituents together preferably entails combining first the ingredients of the water-based lubricant and the homeopathic components. Food grade equipment and conditions can be used in the preparation of the lubricant compositions.

In the case of the rehydrating personal lubricant compositions described above, generally, the deionized water and organic aloe vera juice are combined, then vitamin E is added as a natural preservative, then flavor is added (if included), then grapefruit seed extract is added and finally guar gum is added. The other constituents, e.g., the homeopathic ingredients, can be added at various different times. Everything is mixed in a food grade container. After the mix turns to gel, the mix is pumped into a hopper which can conveniently feed it into bottles or other sealed containers for later distribution and use.

The invention also provides methods of using the personal lubricant compositions described above by applying the lubricant to a female individual prior to sexual activity. The homeopathic components of the lubricant serve to support the health of the female reproductive system.

When the rehydratable personal lubricant formulation is utilized during sexual activity it is effectively rehydrated with the liquids secreted by at least one of the individuals involved. Such native rehydration has been experienced which has provided a nearly unlimited duration of high degree of slipperiness with the use of one of the embodiments of the formulation described herein. In contrast, similar use of personal lubricants derived from petroleum distillates has not been shown to be particularly benefited from any rehydration with bodily secreted liquids. Hence, this native rehydration effect, which provides significant additional duration to the effectiveness of the lubrication, has been found to be uniquely present with the formulation of this invention, and absent from prior art formulations such as those derived from petroleum distillates.

In use and operation, the personal lubricant formulation can be used as follows. The formulation is initially provided in a container which isolates the formulation from the surrounding environment. Typically, a bottle is provided from which the formulation can be squeezed in a relatively controllable fashion. The user then applies the formulation to at least one of the surfaces of one body part of a female individual. As an acceptable alternative, the formulation can be applied to multiple body parts of the female individual. After direct application has occurred, contact between the body part to which the formulation has been applied and another surface, such as a second body part, is initiated with the lubricant located therebetween.

The body part to which the formulation has been applied can be brought into contact with a body part of another individual. As an optional intermediate step, a condom or other additional device can be interposed between the individuals.

After contact is initiated, such contact will typically stimulate the secretion of bodily liquids. When a rehydrating lubricant formula is used, these bodily liquids interact with the formulation in a fashion which rehydrates the formulation and enhances a slipperiness of the formulation by reducing a coefficient of friction exhibited by the formulation. Such rehydration utilizing bodily secreted liquids is referred to as native rehydration. This native rehydration continues to maintain a low coefficient of friction and a high degree of slipperiness of the personal lubricant while the contact between the individuals continues. As a result, a high degree of durational effectiveness is maintained. The individual thus need not interrupt the activity to apply additional lubricant as frequently or require external sources of water or other rehydrating agents to maintain the slipperiness of the personal lubricant. If the individual secretes an insufficient amount of liquid for effective native rehydration, the individual can optionally provide water from a separate source to rehydrate the personal lubricant formulation of this invention.

When the activity ceases, the formulation is easily removed by merely washing off, particularly if the formulation is water based.

The lubricant formulation described herein can reduce the friction between the body part to which the formulation is applied and any other object, such as another body part or a device, such as an a medical device or an instrument, for example, a gynecological medical instrument, douche, enema or tampon. The lubricant formulation can be applied at times other than during or just prior to sexual activity. The lubricant formulation can be used to relieve genital discomfort, such as to relieve discomfort associated with vaginal dryness or infection, or as needed to promote a general feeling of wellbeing on a daily basis. Use of the lubricant formulation can alleviate or reduce post-coital pain and discomfort. The formulation can support the health of the female reproductive system. A formulation that is free from petroleum-based products can be used with a latex-based materials, such as contraceptive devices, for example condoms or diaphragms, or silicon-based materials. Additionally, water-based formulations that include organic ingredients can be safe for ingestion if the individual ingredients are non-toxic. In particular, using low concentrations of homeopathic components can enable formation of a formulation that is safe for ingestion.

The lubricant formulations described herein are capable of native rehydration, and thus the duration of their effectiveness can equal the duration of any sexual activity. Because the body part that is involved in the activity can be protected from friction for the duration of the activity, the likelihood of dryness or inflammation can be reduced. Either or both of the slippery nature of the lubricant formulation and the homeopathic components of the formulation can support the female reproductive system. The formulation can relieve minor vaginal and bladder irritations, such as pain, burning, itching and vaginal discharge. Future occurrences of bladder infections, vaginal itching and candida overgrowth can be prevented.

In some embodiments, the homeopathic ingredients described herein can be incorporated into a formulation appropriate for topical application, such as to the genital region or generally to the body. The formulation can be in any form appropriate for cutaneous application, such as a cream, lotion, gel, emollient, ointment, balm, liquid or oil. In addition to the components described above, the homeopathic ingredients can be combined with excipients, such as oils, waxes, butters, emulsifiers and stabilizers. The formulation can have a liquidity and viscosity that is appropriate for applying and smoothing into the skin. The homeopathic ingredients can be combined with or one or more excipients appropriate for applying to a device, such as a transdermal patch. Some excipients, such as dimethyl sulfoxide (DMSO), can assist in transdermal migration of the homeopathic ingredients. In additional to adding the homeopathic ingredients to water-based formulations, the ingredients can be incorporated into a non-water based formulation, such as a glycerine-, silicon-, or petroleum-based formulation.

### EXAMPLE 1

A female individual with a history of candidiasis, or candida infections, reported that sexual intercourse during an infection was unpleasant. While having a candida infection, the subject applied the rehydrating water-based personal lubricant composition comprising material derived from an aloe vera plant, grapefruit seed extract, vitamin E, guar gum formulation containing pulsatilla 6c, berberis vulgaris 6c, hepar-sulphuris-calcareum 6c, lilium tigrinum 6c, hydrastis canadensis 6c, cantharis 6c and candida albicans 6c prior to sexual intercourse. After sexual intercourse, the subject reported that the candida infection was improved.

This disclosure is provided to reveal a preferred embodiment of the invention and a best mode for practicing the invention. Having thus described the invention in this way, it should be apparent that various different modifications can be made to the preferred embodiment without departing from the scope and spirit of this disclosure.

1. A composition for topical application, comprising at least one component selected from the group consisting of pulsatilla, berberis vulgaris, hepar-sulphuris-calcareum, lilium tigrinum, hydrastis canadensis, cantharis, candida albicans, sepia and thuja occidentalis.

2. The composition of claim 1 which comprises at least one component selected from the group consisting of sepia and thuja occidentalis.

3. The composition of claim 1 which comprises substantially all natural and/or all organic ingredients and components.

4. A water-based personal lubricant composition comprising at least one component selected from the group consisting of pulsatilla, berberis vulgaris, hepar-sulphuris-calcareum, lilium tigrinum, hydrastis canadensis, cantharis, candida albicans, sepia and thuja occidentalis.

5. The composition of claim 4 which comprises at least one component selected from the group consisting of sepia and thuja occidentalis.

6. The composition of claim 4 which comprises substantially all natural and/or all organic ingredients and components.

7. A rehydrating water-based personal lubricant composition comprising at least one component selected from the group consisting of pulsatilla, berberis vulgaris, hepar-sulphuris-calcareum, lilium tigrinum, hydrastis canadensis, cantharis, candida albicans, sepia and thuja occidentalis.

8. The composition of claim 7 which comprises at least one component selected from the group consisting of sepia and thuja occidentalis.

9. The composition of claim 7 which comprises substantially all natural and/or all organic ingredients and components.

10. A rehydrating water-based personal lubricant composition comprising guar gum and at least one component selected from the group consisting of pulsatilla, berberis vulgaris, hepar-sulphuris-calcareum, lilium tigrinum, hydrastis canadensis, cantharis, candida albicans, sepia and thuja occidentalis.

11. A composition of claim 10 which contains at least two components.

12. A composition of claim 10 which contains at least three components.

13. A composition of claim 10 which contains at least four components.

14. A composition of claim 10 which contains at least five components.

15. A composition of claim 10 which contains at least six components.

16. A composition of claim 10 which contains at least seven components.

17. A composition of claim 10 which contains at least eight components.

18. A composition of claim 10 which contains at least nine components.

19. A composition of claim 10 which comprises at least one component selected from the group consisting of sepia and thuja occidentalis.

20. The composition of claim 10 which comprises substantially all natural and/or all organic ingredients and components.

21. The composition of claim 10 further comprising at least one component selected from material derived from an aloe vera plant, grapefruit seed extract or vitamin E.

22. The composition of claim 10 wherein the strength of the homeopathic components used is pulsatilla 6c, berberis vulgaris 6c, hepar-sulphuris-calcareum 6c, lilium tigrinum 6c, hydrastis canadensis 6c, cantharis 6c, candida albicans 6c, sepia 6c, thuja occidentalis 6c.

23. A method of using the personal lubricant composition of claim 10, comprising applying the lubricant composition to a female individual prior to sexual activity.

24. A method of supporting the female reproductive system, comprising administering the composition of claim 1.
